(19) United States
(12) Patent Application Publication
(10) Pub. No.: US 2026/0192334 A1
(43) Pub. Date: Jul. 9, 2026

(54) ACOUSTIC STACKS FOR ULTRASOUND TRANSDUCERS AND METHODS OF MAKING THE SAME

(71) Applicant: GE Precision Healthcare LLC, Waukesha, WI (US)

(72) Inventor: Abhijeet Bhatt, Bangalore (IN)

(73) Assignee: GE Precision Healthcare LLC, Waukesha, WI (US)

(21) Appl. No.: 19/009,720

(22) Filed: Jan. 3, 2025

(51) Int. Cl. B06B 1/06; H10N 30/076

(52) U.S. Cl. CPC B06B 1/067; H10N 30/076; B06B 2201/76

(57) ABSTRACT

Acoustic stacks for ultrasound transducers, and methods of making the same, are provided. In one example, an acoustic stack includes a piezoelectric layer, a matching layer directly disposed on a first side of the piezoelectric layer, and a dematching layer directly disposed on a second side of the piezoelectric layer, the first side opposite the second side. One or more layers may be deposited via thermal spray deposition.

### FIELD

Embodiments of the subject matter disclosed herein relate to ultrasound transducers, and more particularly, to acoustic stacks for ultrasound transducers, and methods of making the same.

### BACKGROUND

Ultrasound imaging systems typically include an ultrasound transducer that performs various ultrasound scans. Ultrasound transducers include one or more acoustic stacks. The acoustic stack transmits ultrasound energy and receives ultrasound signals based on the reflected ultrasound energy. The ultrasound signals received by the acoustic stack are used to generate an image of one or more anatomical structures within a patient.

Acoustic stacks in an ultrasound transducer typically include a piezoelectric material that changes shape in response to the application of a voltage across the piezoelectric material. Changing the potential applied across the piezoelectric material is responsible for generating the ultrasound energy.

### SUMMARY

An embodiment relates to an acoustic stack for an ultrasound transducer. The acoustic stack may include a piezoelectric layer, a matching layer directly disposed on a first side of the piezoelectric layer, and a dematching layer directly disposed on a second side of the piezoelectric layer, the first side opposite the second side.

In another aspect, a method is disclosed. The method includes depositing a matching layer on a first side of a piezoelectric layer via thermal spray deposition.

In another aspect, a method of preparing an acoustic stack is disclosed. The method includes plasma spray depositing a first layer on a substrate, the first layer comprising a composite comprising aluminum, silicon, and graphite; disposing a flex circuit on the first layer; sputtering a second layer on the flex circuit, the second layer comprising gold; electroplating a third layer on the second layer, the third layer comprising nickel; thermal spray depositing a fourth layer on the third layer, the fourth layer comprising tungsten carbide; electroplating a fifth layer on the fourth layer, the fifth layer comprising nickel; thermal spray depositing a sixth layer on the fifth layer, the sixth layer comprising a piezoelectric material; plasma spray depositing a seventh layer on the sixth layer, the seventh layer comprising a composite comprising aluminum, silicon, and graphite; and plasma spray depositing an eighth layer on the seventh layer, the eighth layer comprising polymethyl methacrylate.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a cross-sectional illustration of an acoustic stack for an ultrasound transducer.

FIG. 2 is a cross-sectional illustration of another acoustic stack for an ultrasound transducer.

FIG. 3 is a schematic for a process for preparing the acoustic stack in FIG. 1.

FIG. 4 is a schematic for another process for preparing the acoustic stack in FIG. 1.

FIG. 5 is a schematic for a process for preparing the acoustic stack in FIG. 2.

FIG. 6 is a schematic for another process for preparing the acoustic stack in FIG. 2.

FIG. 7 is a schematic for a process for preparing a diced acoustic stack.

FIG. 8 is a schematic for another process for preparing a diced acoustic stack.

FIG. 9 is a schematic for another process for preparing a diced acoustic stack.

FIG. 10 is a cross-sectional illustration of an ultrasound probe with an acoustic stack.

### DETAILED DESCRIPTION

Referring generally to the figures, acoustic stacks for an ultrasound transducers are disclosed. An acoustic stack includes a piezoelectric layer, a matching layer directly disposed on one side of the piezoelectric layer, and a dematching layer directly disposed on another side of the piezoelectric layer opposite the side of the piezoelectric layer with the matching layer.

Conventionally, acoustic stacks are prepared by bonding the matching layer and the dematching layer to the piezoelectric layer using an adhesive (e.g., epoxy), which may be sintered. Typically, thicker layers of layer material are bonded to the piezoelectric layer for ease of handling and, if applicable, to accommodate sintering, followed by grinding or lapping of the layers to a predetermined thickness to form the matching and dematching layers useful in the acoustic stack. Good matching characteristics may be achieved if the thickness of the graded matching layer is in the range of about one quarter wavelength to about two wavelengths. For example, where sintering is used to bond the matching layer to the piezoelectric layer, the matching layer may have a thickness of about 2.5 mm or greater to maintain layer flatness during sintering, and the matching layer may be ground down to a thickness of about 50 µm to about 250 µm. Given the tight tolerances used for effective acoustic stacks, the grinding or lapping processes complicate large-scale manufacturing of acoustic stacks. In particular, these processes can consume significant volumes of water and create environmental waste, increasing cost and complexity.

To address these problems, disclosed herein are acoustic stacks for ultrasound transducers and methods of making acoustic stacks using layer deposition processes that directly deposit layers of the acoustic stack upon one another such that layers are directly disposed on one another without the use of adhesive and sintering between layers. Avoiding the use of adhesive may improve acoustic stack performance by reducing acoustic impedance mismatch, thereby increasing resolution and sensitivity. Furthermore, these processes may deposit micron-scale layers precisely, avoiding or substantially reducing the need for grinding or lapping to create a layer with a predetermined thickness, thereby avoiding manufacturing complications related to grinding or lapping.

Layer deposition processes used to form the layers of the acoustic stack disclosed herein include thermal spray deposition, including plasma spraying, detonation spraying, wire arc spraying, flame spraying, high velocity oxy-fuel (HVOF) spraying, high velocity air fuel (HVAF) spraying, and warm spraying. Layer deposition processes may further include cold spraying, physical vapor deposition (PVD), chemical vapor deposition (CVD), atomic layer deposition (ALD), pulsed laser deposition (PLD), molecular beam epitaxy (MBE), or electroplating. For example, seed layers may be formed using spray deposition, PVD, CVD, or electroplating to facilitate the formation of thermal spray-deposited matching and dematching layers.

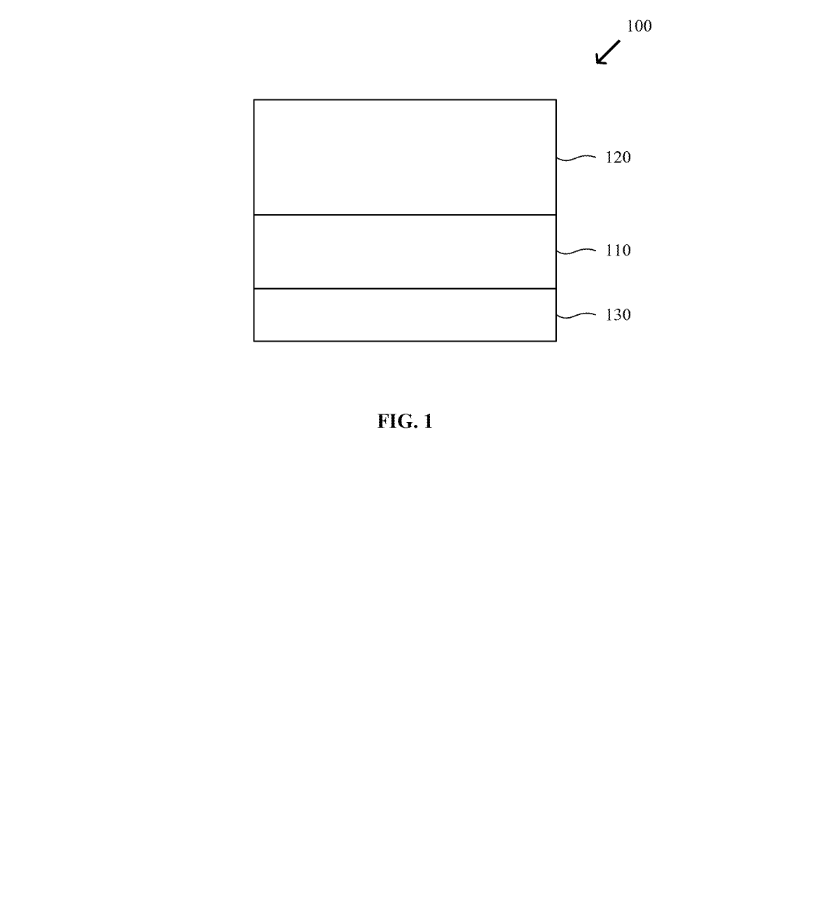

FIG. 1 is a cross-sectional illustration of an acoustic stack 100 for an ultrasound transducer. The acoustic stack 100 includes a piezoelectric layer 110 with a matching layer 120 disposed on one side of the piezoelectric layer 110 and a dematching layer 130 directly disposed on the opposite side of the piezoelectric layer 110. The matching layer 120, the dematching layer 130, and optionally the piezoelectric layer 110, are deposited using the processes disclosed herein. The acoustic stack 100 may be free of adhesive between the different layers. Instead, the matching layer 120 and the dematching layer 130 may be mechanically and/or chemically bonded to the piezoelectric layer 110, where mechanical bonding results from the layer deposition processes used to form the acoustic stack layers.

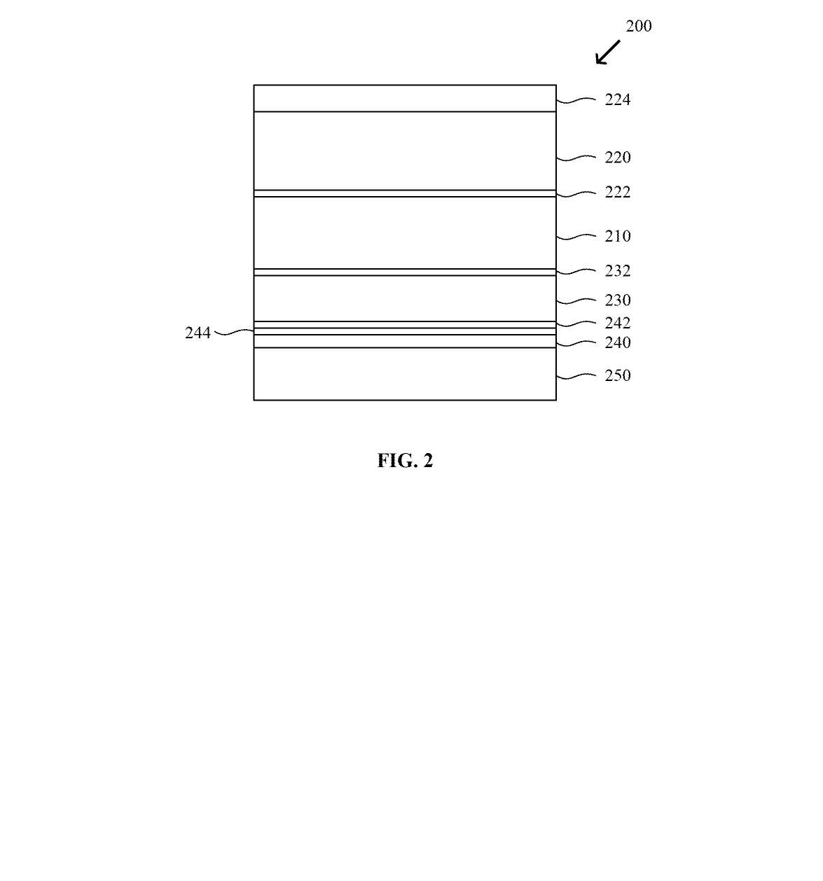

FIG. 2 is a cross-sectional illustration of another acoustic stack 200 for an ultrasound transducer. The acoustic stack 200 includes a piezoelectric layer 210, a first matching layer 220 disposed on one side of the piezoelectric layer 210, and a dematching layer 230 disposed on the other side of the piezoelectric layer 210. Between the piezoelectric layer 210 and the first matching layer 220 is a matching seed layer 222 to facilitate deposition of the first matching layer. Disposed on the first matching layer 220 is a second matching layer 224. Between the piezoelectric layer 210 and the dematching layer 230 is a dematching seed layer 232 to facilitate deposition of the dematching layer 230. A conductive metal layer 244 is disposed on the dematching layer 230, with a conductive metal seed layer 242 therebetween. The flexible circuit 240 is disposed on the conductive metal layer 244. The flexible circuit 240 is disposed on a substrate 250.

In any embodiment, the piezoelectric layer may be configured to generate and transmit acoustic energy into a patient (not shown) and receive backscattered acoustic signals from the patient to create and display an image. The piezoelectric layer typically has an acoustic impedance of around 37 MRayls and human tissue typically has an acoustic impedance of around 1.5 MRayls. The piezoelectric layer may include electrodes on the top and bottom surfaces, as known in the art.

The piezoelectric layer may be formed of a piezoelectric ceramic, a piezocomposite, a piezoelectric single crystal, or a piezopolymer. Examples of piezoelectric ceramic materials include, but are not limited to, lead zirconate titanate (PZT), lead magnesium niobate-lead titanate (PMN-PT), lead indium niobate-lead magnesium niobate-lead titanite (PIN-PMN-PT), magnesium-doped PIN-PMN-PT, lead magnesium niobate, lead titanate, lead indium niobate, barium titanate, lithium niobate, lithium tantalate, aluminum scandium nitride, silicon carbide, or combinations of two or more thereof. The piezoelectric layer may include multiple layers of the aforementioned materials. The piezoelectric layer may include multiple layers of the same material or may include multiple layers of different materials.

The piezoelectric layer may have a thickness of about 0.01 mm to about 2 mm. Different thicknesses of the piezoelectric layer may be used for low-frequency (e.g., about 1 MHz to about 5 MHz) and high-frequency (e.g., about 5 MHz to about 10 MHz) transducers. Acoustic stacks for low frequency transducers may include piezoelectric layers having a thickness of about 0.5 mm to about 2 mm (e.g., about 0.5 mm to about 1 mm, about 0.5 mm, about 0.6 mm, about 0.7 mm, about 0.8 mm, about 0.9 mm, or about 1 mm). Acoustic stacks for high frequency transducers may include piezoelectric layers having a thickness of about 0.01 mm to about 0.5 mm (e.g., about 0.02 mm to about 0.5 mm, about 0.05 mm to about 0.5 mm, or about 0.1 mm to about 0.5 mm, about 0.01 mm, about 0.02 mm, about 0.03 mm, about 0.04 mm, about 0.05 mm, about 0.06 mm, about 0.07 mm, about 0.08 mm, about 0.09 mm, about 0.1 mm, about 0.2 mm, about 0.3 mm, about 0.4 mm, or about 0.5 mm).

In any embodiment, one or more matching layers may facilitate matching of an impedance differential that may exist between the piezoelectric layer and a patient. Any number of matching layers may be provided. Each matching layer may have any value of acoustic impedance, such as, but not limited to, between approximately 2 MRayls and approximately 15 MRayls and/or less than approximately 10 MRayls. In some embodiments, each matching layer has an acoustic impedance that is less than the acoustic impedance of the piezoelectric layer. In some embodiments, a plurality of matching layers is provided that provide a progressive reduction in acoustic impedance from the piezoelectric layer. For example, in some embodiments, three matching layers are provided, wherein the matching layer closest to the piezoelectric layer is approximately 15 MRayls, the next matching layer is approximately 8 MRayls, and the matching layer farthest from the piezoelectric layer is approximately 3 MRayls. In some embodiments, one or more matching layers include a gradient of acoustic impedance.

Each matching layer may be electrically conductive or electrically non-conductive. When a matching layer is electrically non-conductive, the matching layer may include a conductive film layer (not shown) thereon. One or more matching layers (and/or a conductive film layer thereon) may provide an electrical ground connection for the corresponding electrode between the dematching layer and the flexible circuit, as described herein.

The one or more matching layers may be formed of graphite, aluminum, silicon, polymer, or a combination of two or more thereof. The one or more matching layers may include metal silicon carbon composite materials (e.g., aluminum silicon graphite composites, aluminum silicon polymer composites, and combinations thereof), polymers, and combinations thereof. Exemplary polymers that may be present in the matching layers include, but are not limited to, polymethylmethacrylate (PMMA), polyester, polyethylene terephthalate (PET), polyvinylidene fluoride (PVDF), and mixtures or copolymers of any two or more thereof. For example, the matching layer may include one or more layers formed of aluminum silicon graphite disposed closer to the piezoelectric layer, and one or more polymer layers (e.g., polyester or PMMA) disposed on the aluminum silicon graphite layers further from the piezoelectric layer. For example, the matching layer may be formed of two layers with one layer comprising graphite, aluminum, silicon, polymer, or a combination of two or more thereof (e.g., an aluminum silicon carbon composites), and a second layer comprising the polymer. As another example, the matching layer may be formed of two layers with one layer comprising the aluminum silicon graphite composite and another layer comprising the aluminum silicon polymer composite.

The aluminum silicon carbon composites may include about 1 wt.% to about 20 wt.% silicon, e.g., about 1 wt.% to about 10 wt.%, about 2 wt.% to about 8 wt.%, about 1 wt.%, about 2 wt.%, about 3 wt.%, about 4 wt.%, about 5 wt.%, about 6 wt.%, about 7 wt.%, about 8 wt.%, about 9 wt.%, about 10 wt.%, about 15 wt.%, or about 20 wt.% relative to the total weight of the composite.

The aluminum silicon carbon composites may include about 40 wt.% to about 80 wt.% aluminum, e.g., about 45 wt.% to about 75 wt.%, about 50 wt.% to about 70 wt.%, about 50 wt.% to about 60 wt.%, about 50 wt.%, about 55 wt.%, about 60 wt.%, about 65 wt.%, about 70 wt.%, about 75 wt.%, or about 80 wt.% relative to the total weight of the composite.

The aluminum silicon graphite composites may include about 10 wt.% to about 60 wt.% graphite, e.g., about 20 wt.% to about 50 wt.%, about 20 wt.% to about 30 wt.%, about 40 wt.% to about 50 wt.%, about 20 wt.%, about 22 wt.%, about 24 wt.%, about 26 wt.%, about 28 wt.%, about 30 wt.%, about 32 wt.%, about 34 wt.%, about 36 wt.%, about 38 wt.%, about 40 wt.%, about 42 wt.%, about 44 wt.%, about 46 wt.%, about 48 wt.%, about 50 wt.%, about 52 wt.%, about 54 wt.%, about 56 wt.%, about 58 wt.%, or about 60 wt.% ± 1 wt.% relative to the total weight of the composite.

The aluminum silicon graphite may have a gradient composition with a progressively increasing weight percentage of graphite with distance from the piezoelectric layer that provides a progressive reduction in acoustic impedance from the piezoelectric layer. For example, the gradient composite may include graphite in an amount of about 10 wt.% to about 30 wt.% on the side of the composite closer to the piezoelectric layer and about 35 wt.% to about 55 wt.% on the side of the composite further from the piezoelectric layer, with a graded change in graphite concentration over the thickness of the layer (e.g., linear grade or polynomial grade).

The aluminum silicon polymer composites may include about 10 wt.% to about 60 wt.% polymer, e.g., about 20 wt.% to about 50 wt.%, about 20 wt.% to about 30 wt.%, about 40 wt.% to about 50 wt.%, about 20 wt.%, about 22 wt.%, about 24 wt.%, about 26 wt.%, about 28 wt.%, about 30 wt.%, about 32 wt.%, about 34 wt.%, about 36 wt.%, about 38 wt.%, about 40 wt.%, about 42 wt.%, about 44 wt.%, about 46 wt.%, about 48 wt.%, about 50 wt.%, about 52 wt.%, about 54 wt.%, about 56 wt.%, about 58 wt.%, or about 60 wt.% ± 1 wt.% relative to the total weight of the composite.

Each matching layer may have any thickness and the matching layers may have any combined thickness. Examples of the combined thickness of the matching layers include, but are not limited to, a thickness of approximately relative to the wavelength (λ) at the resonant frequency of about λ/k, where k is 1, 2, or 4. Depending on the resonant frequency, the combined thickness of the matching layers may be about 1 µm to about 1000 µm (e.g., about 2 µm to about 20 µm, about 5 µm to about 500 µm, about 10 µm to about 200 µm, or about 20 µm to about 100 µm). Any matching layer may have a thickness of about 1 µm to about 500 µm (e.g., about 2 µm to about 20 µm, about 4 µm to about 100 µm, about 10 µm to about 50 µm, or about 20 µm to about 40 µm).

Between the piezoelectric layer and the matching layer may be a matching seed layer to facilitate deposition of the first matching layer on the piezoelectric layer or vice versa. The seed layer may provide nucleation sites for the deposition of the matching layer on the piezoelectric layer or provide nucleation sites for the deposition of the piezoelectric layer on the matching layer. The seed layer may be formed of gold, nickel, aluminum, aluminum oxide, tungsten carbide, graphite, silicon, silicon oxide, or a combination of any two or more thereof. For example, the seed layer may be a silicon aluminum graphite composite. The seed layer may be a continuous layer or a noncontinuous layer, depending on the thickness of the layer and type of deposition used to form the layer. The seed layer may have a thickness of about 0.1 µm to about 10 µm (e.g., 0.1 µm to about 5 µm, about 0.3 µm to about 5 µm, or about 0.5 µm to about 2 µm).

Where the acoustic stack includes more than one matching layer, the acoustic stack may include additional seed layers between matching layers. The additional seed layers may be formed of gold, nickel, aluminum, aluminum oxide, tungsten carbide, graphite, silicon, silicon oxide, or a combination of any two or more thereof. For example, the additional seed layers may be silicon aluminum graphite composites. The additional seed layers may be continuous layers or noncontinuous layers, for example, depending on the thickness of the layer and type of deposition used to deposit the layer. The seed layers may have a thickness of about 0.1 µm to about 10 µm (e.g., 0.1 µm to about 5 µm, about 0.3 µm to about 5 µm, or about 0.5 µm to about 2 µm).

Between the piezoelectric layer and the dematching layer may be a dematching seed layer to facilitate deposition of the dematching layer or the piezoelectric layer. The seed layer may provide nucleation sites for the deposition of the dematching layer on the piezoelectric layer or provide nucleation sites for the deposition of the piezoelectric layer on the dematching layer. The seed layer may be formed of gold, nickel, aluminum, tungsten carbide, silicon, aluminum oxide, silicon oxide, or a combination of any two or more thereof. The seed layer may be a continuous layer or a noncontinuous layer, depending on the thickness of the layer and type of deposition used to form the layer. The seed layer may have a thickness of about 0.1 µm to about 10 µm (e.g., 0.1 µm to about 5 µm, about 0.3 µm to about 5 µm, or about 0.5 µm to about 2 µm).

In any embodiment, one or more dematching layers may be included in the acoustic stack. A dematching layer may be a layer having a higher acoustic impedance than the piezoelectric layer disposed between the piezoelectric layer and the flexible circuit. The dematching layer may reduce acoustic artifacts. For example, the acoustic impedance of the piezoelectric layer may be in a range from about 3 MRayls to about 35 MRayls, while the acoustic impedance of the dematching layer may be in a range from about 60 MRayls to about 100 MRayls (e.g., above 70 MRayls). The dematching layer may be formed of tungsten carbide, tungsten, tantalum, or other materials with similar acoustic impedance, or a combination of any two or more thereof. The dematching layer functions as an acoustic impedance transformer, dramatically increasing the effective acoustic impedance presented at (or experienced by) the rear face of the piezoelectric layer to a value substantially greater than the impedance of the piezoelectric layer. Consequently, a majority of the acoustic energy is reflected out a front face of the piezoelectric layer.

The dematching layer may include one or multiple layers. The combined thickness of the layers of the dematching layer may be about 1 µm to about 500 µm (e.g., about 2 µm to about 20 µm, about 4 µm to about 100 µm, about 10 µm to about 50 µm, about 20 µm to about 40 µm, about 2 µm, 4 µm, 6 µm 8 µm, 10 µm, 20 µm, 30 µm, 40 µm, 50 µm, 60 µm, 70 µm, 80 µm, 90 µm, 100 µm, 200 µm, 300 µm, 400 µm, or 500 µm. Any dematching layer may have a thickness of about 1 µm to about 500 µm (e.g., about 2 µm to about 20 µm, about 4 µm to about 100 µm, about 10 µm to about 50 µm, about 20 µm to about 40 µm, about 2 µm, 4 µm, 6 µm 8 µm, 10 µm, 20 µm, 30 µm, 40 µm, 50 µm, 60 µm, 70 µm, 80 µm, 90 µm, 100 µm, 200 µm, 300 µm, 400 µm, or 500 µm).

The acoustic stack may include a conductive metal layer disposed between the dematching layer and the flexible circuit. The conductive metal layer may be an electrode. During operation of the ultrasound transducer, an electrical waveform pulse may be applied to the electrode, causing a mechanical change in the piezoelectric layer, and generating an ultrasound wave. Nonlimiting examples of metals that may be used to form the conductive metal layer include gold, platinum, silver, nickel, aluminum, copper, steel, or a combination of any two or more thereof. The conductive metal layer may have a thickness of about 0.05 mm to about 2 mm (e.g., about 0.05 mm to about 1 mm, about 0.05 mm, about 0.5 mm, about 0.05 mm, about 0.4 mm, about 0.05 mm, or about 0.1 mm).

The acoustic stack may include a seed layer between the conductive metal layer and the dematching layer. The seed layer may provide nucleation sites for the deposition of the dematching layer on the conductive metal layer or provide nucleation sites for the deposition of the conductive metal layer on the dematching layer. The seed layer may be formed of gold, nickel, aluminum, tungsten carbide, tungsten, silicon, silicon oxide, aluminum oxide, or a combination of any two or more thereof. The seed layer may be a continuous layer or a noncontinuous layer, depending on the thickness of the layer and type of deposition used to form the layer. The seed layer may have a thickness of about 0.1 µm to about 10 µm (e.g., 0.1 µm to about 5 µm, about 0.3 µm to about 5 µm, or about 0.5 µm to about 2 µm).

The flexible circuit may be disposed on the conductive metal layer. The flexible circuit is an integrated circuit. The integrated circuit may be any type of integrated circuit, such as, but not limited to, an application specific integrated circuit (ASIC) and/or the like. Various components of an ultrasound system may be included within the integrated circuit. For example, the integrated circuit may include a transmitter, a receiver, and beamforming electronics of the ultrasound system.

The flexible circuit may be disposed on a substrate. The substrate may facilitate fabrication of the acoustic stack. For example, the substrate may act as a surface on which the layers of the acoustic stack are formed. The substrate may be formed of a solid material that is stable at temperatures of at least 500 °C (e.g., steel, titanium, tungsten, silicon). The substrate may include a surface layer acting as an acoustic backing layer. The surface layer may be formed of graphite, aluminum, silicon, or a combination of two or more thereof. For example, the surface layer may include an aluminum silicon graphite composite.

Another aspect of the present technology includes methods of forming acoustic stacks. These methods may use layer deposition processes, such as, but not limited to thermal spray deposition (e.g., plasma spraying, detonation spraying, wire arc spraying, flame spraying, high velocity oxy-fuel (HVOF) spraying, high velocity air fuel (HVAF) spraying, and warm spraying), cold spraying, physical vapor deposition (PVD), chemical vapor deposition (CVD), atomic layer deposition (ALD), pulsed laser deposition (PLD), molecular beam epitaxy (MBE), electroplating, and any combination of two or more thereof, to deposit the layers of the acoustic stack.

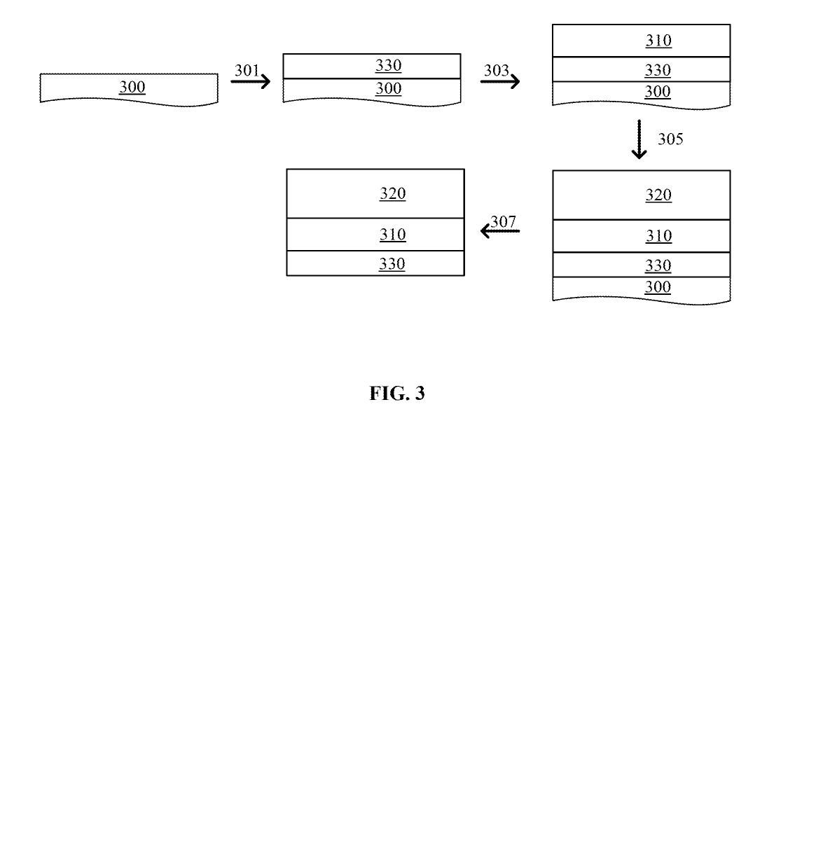

FIG. 3 is a schematic for a process for preparing the acoustic stack in FIG. 1. This process uses a full stack processing approach. In step 301, the dematching layer 330 is deposited on the substrate 300. In step 303, the piezoelectric layer 310 is deposited on the dematching layer 330. In step 305, the matching layer 320 is deposited on the piezoelectric layer 310, forming the acoustic stack. In step 307, the acoustic stack may optionally be removed from the substrate 300. In other embodiments, the order of deposition may be reversed, with the matching layer deposited on the substrate, the piezoelectric layer deposited on the matching layer, and the dematching layer deposited on the piezoelectric layer.

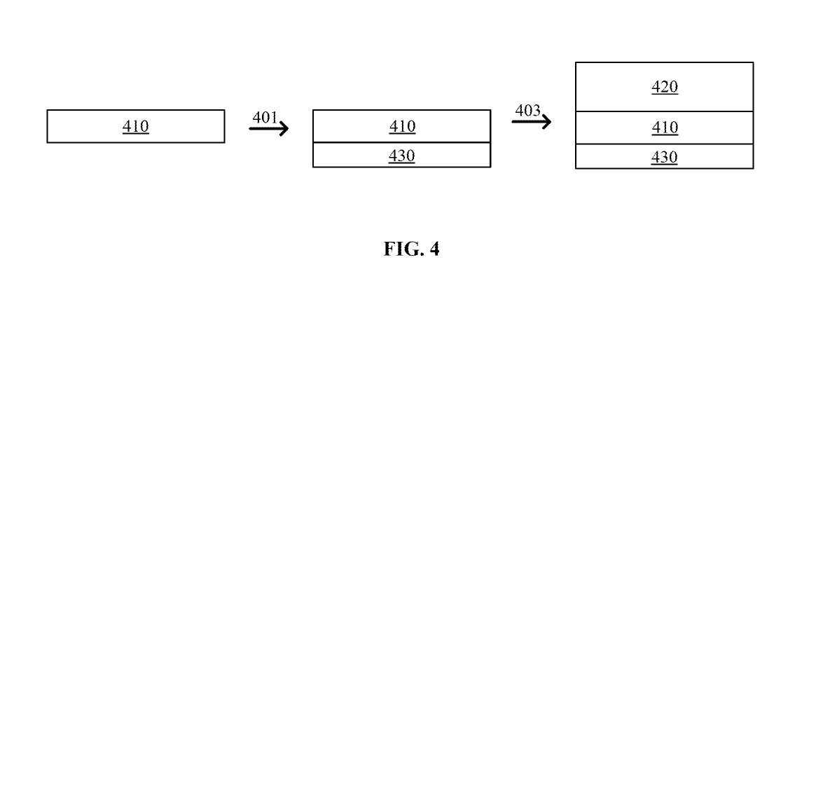

FIG. 4 is a schematic for another process for preparing the acoustic stack in FIG. 1. This process uses a front-end and back-end processing approach. In step 401, the dematching layer 430 is deposited on the piezoelectric layer 410. In step 403, the matching layer 420 is deposited on the piezoelectric layer 410. In other embodiments, the order may be reversed, with the matching layer deposited on the piezoelectric layer and then the dematching layer deposited on the piezoelectric layer.

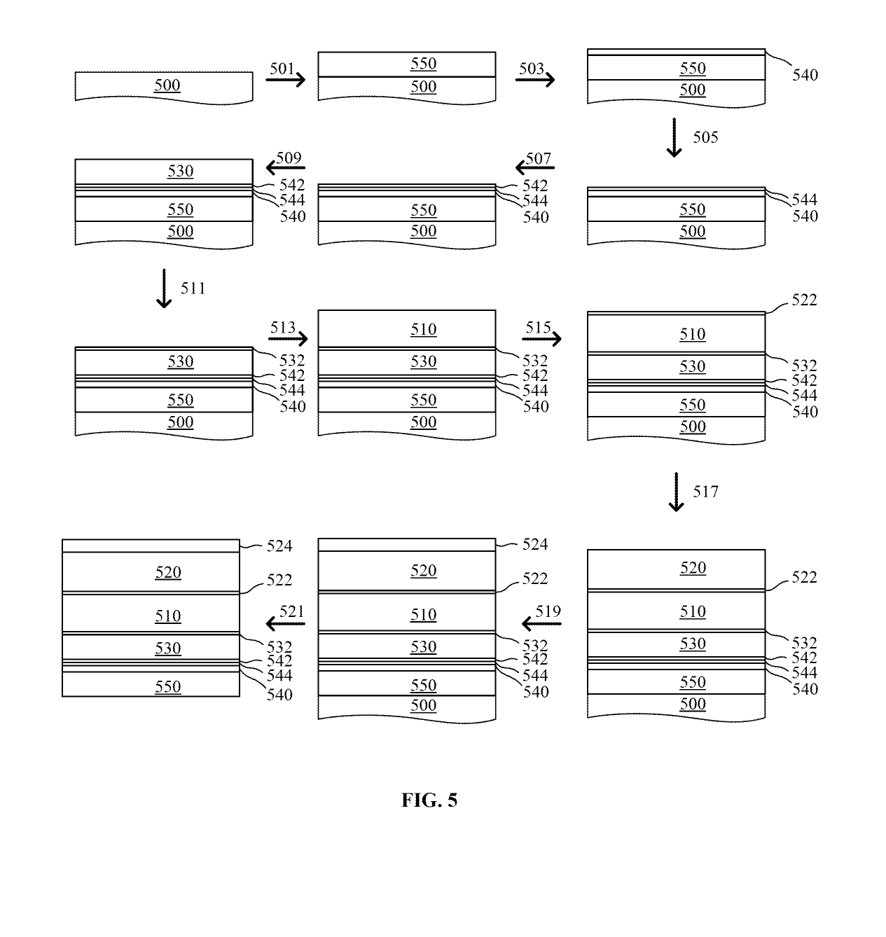

FIG. 5 is a schematic for a process for preparing the acoustic stack in FIG. 2. This process uses a full stack processing approach. In step 501, the backing acoustic layer 550 may be deposited on the substrate 500. In step 503, the flexible circuit 540 may be disposed on the backing acoustic layer 550. In step 505, the conductive metal layer 544 is deposited on the flexible circuit 540. In step 507, the dematching seed layer 542 is deposited on the conductive metal layer 544. In step 509, the dematching layer 530 may be deposited on the dematching seed layer 542. In step 511, the piezoelectric seed layer 532 is deposited on the dematching layer 530. In step 513, the piezoelectric layer 510 is deposited on the piezoelectric seed layer 532. In step 515, the matching seed layer 522 is deposited on the piezoelectric layer 510. In step 517, the first matching layer 520 is deposited on the matching seed layer 522. In step 519, the second matching layer 524 is deposited on the first matching layer 520. In other embodiments, the order of deposition in FIG. 5 may be reversed.

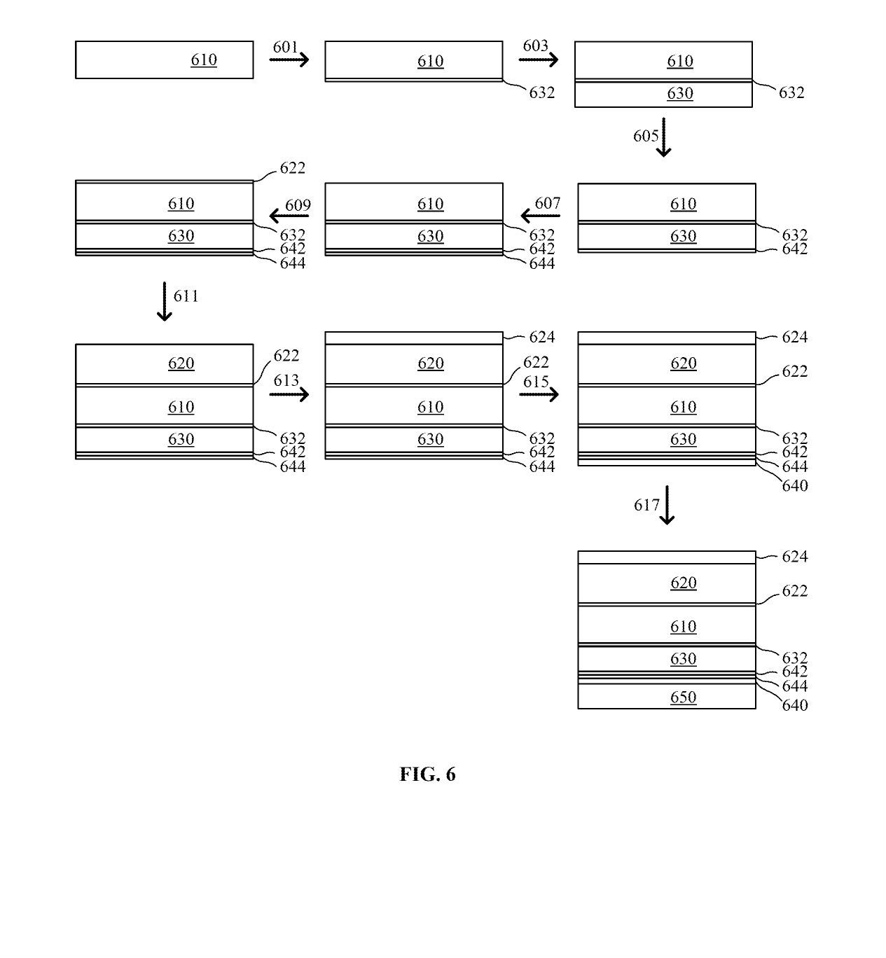

FIG. 6 is a schematic for another process for preparing the acoustic stack in FIG. 2. This process uses a front-end and back-end processing approach. In step 601, the dematching seed layer 632 is deposited on the piezoelectric layer 610. In step 603, the dematching layer 630 is deposited on the dematching seed layer 632. In step 605, the conductive seed layer 642 is deposited on the dematching layer 630. In step 607, the conductive layer 644 is deposited on the conductive seed layer 642. In step 609, the matching seed layer 622 is deposited on a surface of the piezoelectric layer 610 opposite the surface of the piezoelectric layer 610 upon which the dematching seed layer 632 is disposed. In step 611, the first matching layer 620 is deposited on the matching seed layer 622. In step 613, the second matching layer 624 is deposited on the first matching layer 620. In step 615, the flex circuit 640 is disposed on the conductive layer 644. In step 617, the backing acoustic layer 650 is deposited on the flex circuit 640. In other embodiments, the order of deposition in FIG. 6 may be changed, for example with the matching layers deposited before the dematching layer.

In any embodiment of the processes disclosed herein, one or more seed layer may or may not be deposited to facilitate deposition of the the matching layers, dematching layers, piezoelectric layers, conductive layers, and backing acoustic layers, depending on the type of deposition used to deposit the layers and the type of material deposited.

The layers of the acoustic stack may be applied, deposited, or otherwise formed by any of a variety of conventional techniques, including thermal spray deposition methods (e.g., plasma spraying, detonation spraying, wire arc spraying, flame spraying, high velocity oxy-fuel (HVOF) spraying, high velocity air fuel (HVAF) spraying, and warm spraying), cold spraying, physical vapor deposition (PVD) (e.g., electron beam physical vapor deposition (EBPVD), plasma spray, including air plasma spray (APS) and vacuum plasma spray (VPS)), chemical vapor deposition (CVD), atomic layer deposition (ALD), pulsed laser deposition (PLD), molecular beam epitaxy (MBE), electroplating, or combinations of such techniques, such as, for example, a combination of thermal spray, electroplating, and PVD or CVD techniques. For example, thermal spraying may be used to form one or more of the matching layers, dematching layers, piezoelectric layers, conductive layers, backing acoustic layers, and seed layers. Seed layers may be formed using PVD and/or CVD. In some embodiments, the conductive layer may be formed using electroplating. An advantageous aspect of these deposition techniques is the ability to deposit a layer to a predetermined thickness without or with substantially reduced amounts of grinding or lapping. An advantage of thermal spray deposition techniques is that the layers may be deposited under ambient conditions and may not require a vacuum or an explosion-proof environment. Moreover, no curing or additional process steps may be needed. These deposition techniques may be used to form acoustic stacks in batch or continuous processes.

Various types of thermal spray techniques well known to those skilled in the art can be used to apply or deposit one or more layers of the acoustic stack. During thermal spraying processes, the substrate temperature may advantageously remain in a temperature range of about 20 °C to about 100 °C or about 20 °C to about 80 °C or about 20 °C to about 70 °C or about 20 °C to about 60 °C or about 20 °C to about 50 °C. Thermal spraying is particularly suited for depositing thin films having a thickness of about 10 µm to about 10 mm.

Feedstocks for thermal spray techniques may include particles having an average diameter as measured by SEM of about 10 µm to about 500 µm, about 10 µm to about 250 µm, or about 10 µm to about 100 µm. For forming an aluminum silicon graphite composite matching layer with thermal spraying, the feedstock may include particles of aluminum alloy graphite composite powders (e.g. about 30 wt.% to about 80 wt.% aluminum, about 1 wt.% to about 20 wt.% silicon, and about 10 wt.% to about 60 wt.% graphite, as measured by the total weight of the composite powder, or in a weight ratio of aluminum:silicon:graphite of about 64:7:22, 61:6:24, or 42:5:45). For forming an aluminum silicon polyester composite matching layer, the feedstock may include powders of aluminum silicon metallic matrix with polymer (e.g., polyester) embedded as a dislocated/weakening phase (e.g., about 30 wt.% to about 80 wt.% aluminum, about 1 wt.% to about 20 wt.% silicon, and about 10 wt.% to about 60 wt.% graphite, as measured by the total weight of the composite powder, or in a weight ratio of aluminum:silicon:polymer of about 53:7:40).

Plasma spray techniques involve the formation of high-temperature plasma, which produces a thermal plume. See, for example, Kirk-Othmer Encyclopedia of Chemical Technology, 3rd Ed., Vol. 15, page 255, and references noted therein, as well as U.S. Pat. No. 5,332,598 (Kawasaki et al), issued Jul. 26, 1994; U.S. Pat. No. 5,047,612 (Savkar et al) issued Sep. 10, 1991; and U.S. Pat. No. 4,741,286 (Itoh et al), issued May 3, 1998, which are instructive in regard to various aspects of plasma spraying suitable for use herein. Plasma spray techniques may be used to deposit, for example, metals, metal carbides, metal oxides, silicon, silicon oxide, and metal alloys. The acoustic stack layer materials, e.g., tungsten carbide powder for depositing a tungsten carbide dematching layer, or aluminium silicon graphite powders for depositing an aluminum silicon graphite matching layer, are fed into the plume, and the high-velocity plume is directed toward the surface of piezoelectric layer. Various details of such plasma spray coating techniques will be well-known to those skilled in art, including various relevant steps and process parameters such as plasma spray parameters such as spray distances (gun-to-substrate), selection of the number of spray-passes, powder feed rates, particle velocity, torch power, plasma gas selection, oxidation control to adjust oxide stoichiometry, angle-of-deposition, post-treatment of the applied coating; and the like. Torch power may vary in the range of about 10 kilowatts to about 200 kilowatts, for example, ranges from about 40 kilowatts to about 60 kilowatts. The velocity of the dematching layer coating material particles flowing into the plasma plume (or plasma “jet”) is another parameter that may be closely controlled.

Briefly, a typical plasma spray system includes a plasma gun anode which has a nozzle pointed in the direction of the deposit-surface of the substrate being coated. The plasma gun is often controlled automatically, e.g., by a robotic mechanism, which is capable of moving the gun in various patterns across the substrate surface. The plasma plume extends in an axial direction between the exit of the plasma gun anode and the substrate surface. Some sort of powder injection means is disposed at a predetermined, desired axial location between the anode and the substrate surface. In some embodiments of such systems, the powder injection means is spaced apart in a radial sense from the plasma plume region, and an injector tube for the powder material is situated in a position so that it can direct the powder into the plasma plume at a desired angle. The powder particles, entrained in a carrier gas, are propelled through the injector and into the plasma plume. The particles are then heated in the plasma and propelled toward the substrate. The particles melt, impact on the substrate, and quickly cool to form the layer of the acoustic stack.

Wire arc spraying, also called plasma transferred wire arc (PTWA) thermal spraying is a form of plasma spraying that uses a conductive wire as feedstock for the plasma spray system. As an example, wire arc spraying may be used to form the conductive metal layer of the acoustic stack. Briefly, wire arc spraying includes a supersonic plasma jet, formed by a transferred arc between a non-consumable cathode and the wire, which melts and atomizes the wire. The atomized wire particles are entrained in a carrier gas and are propelled toward the substrate, where the particles flatten upon striking the substrate due to their high kinetic energy and rapidly solidify, forming the layer on the surface. Various details of such wire arc spraying techniques will be well-known to those skilled in art, including various relevant steps and process parameters such as plasma spray parameters such as spray distances (gun-to-substrate), selection of the number of spray-passes, type and thickness of the wire, particle velocity, plasma gas selection, angle-of-deposition, post-treatment of the applied coating; and the like.

A suitable thermal spray method for depositing thin, dense, and smooth layers of the acoustic stack is by high velocity oxygen flame (HVOF) or high velocity air fuel spray (HVAF). HVOF and HVAF may be used to deposit, for example, metals, metal carbides, metal oxides, silicon, silicon oxide, silicon carbide, and metal alloys. By way of example, HVOF or HVAF may be used deposit the tungsten carbide dematching layer or the aluminum silicon graphite composite matching layer. As another example, HVOF or HVAF may be used to deposit the piezoelectric layer formed of lead zirconate titanate (PZT), lead magnesium niobate-lead titanate (PMN-PT), lead indium niobate-lead magnesium niobate-lead titanite (PIN-PMN-PT), magnesium-doped PIN-PMN-PT, lead magnesium niobate, lead titanate, lead indium niobate, barium titanate, lithium niobate, lithium tantalate, aluminum scandium nitride, silicon carbide, or a combination of any two or more thereof. The heat source may be, respectively, a flame or a thermal plume controlled by the input gases, fuels, and nozzle designs. Oxygen or air fuel are supplied at high pressure such that the flame issues from the nozzle at supersonic velocity. A gun having a convergent/divergent or straight bore nozzle can be used to apply the dematching layer. A person with skill in the art would know how to adjust these process parameters.

A modification of HVOF that may be used to deposit layers of the acoustic stack is warm spraying. Warm spraying may be used to deposit, for example, metals, metal carbides, metal oxides, metal alloys, and any combination of any two or more thereof from feedstock powders. By injecting inert gas at a lower temperature (e.g., about 20 °C to about 30 °C) into the combustion gas jet of HVOF, the temperature of the propellant gas can be controlled in a range approximately from about 700 °C to about 2000 °C so that many powder materials can be deposited in thermally softened state at high impact velocity. Deposition parameters, including amount of lower temperature gas mixed into the carrier gas, heat source, carrier gas velocity, nozzle designs, and the stoichiometric ratio of the powder mixture may be changed to achieve predetermined layer characteristics. A person with skill in the art would know how to adjust these process parameters.

Another suitable thermal spray method for depositing thin, dense, and smooth layers of the acoustic stack is by flame spraying. Flame spraying may be used to deposit, for example, metals, metal carbides, metal oxides, metal alloys, polymers, and any combination of any two or more thereof from feedstock powders, rods, or wires. By way of example, detonation spraying may be used to deposit a tungsten carbide dematching layer. Briefly, in this process, a combustible gas, such as acetylene or propane, is ignited with oxygen to produce a high-temperature flame that melts the feedstock into particles. These molten particles are then propelled onto the substrate, forming a robust coating. The flame parameters, including the mixture ratio of gas to oxygen and the feed rate of the material, can be adjusted to change the deposition quality and layer properties. A person with skill in the art would know how to adjust these process parameters.

Another suitable thermal spray method for depositing thin, dense, and smooth layers of the acoustic stack is by detonation spraying. Detonation spraying may be used to deposit, for example, metals, metal carbides, metal oxides, metal alloys, silicon, silicon oxide, and any combination of any two or more thereof from feedstock powders. By way of example, detonation spraying may be used to deposit a tungsten carbide dematching layer. Briefly, in this method, a controlled detonation of a fuel-air mixture generates high-pressure and high-temperature gases, which propel the feedstock particles at supersonic velocities towards the substrate. See, for example, U.S. Pat. No. 2,714,563 (Poorman et al.), issued Aug. 2, 1955. The process can be finely tuned by varying parameters such as the type of fuel, the stoichiometric ratio of the powder mixture, and the geometry of the detonation chamber. A person with skill in the art would know how to adjust these process parameters.

Cold spraying is another spraying technique that may be used to deposit layers of the acoustic stack. Cold spraying may be used to deposit, for example, metals, metal carbides, metal oxides, metal alloys, polymers, and any combination of any two or more thereof from feedstock powders. By way of example, cold spraying may be used to deposit a tungsten carbide dematching layer. Briefly, in this process, solid powders are entrained in a carrier gas and propelled at a velocity of about 350 m/s to about 1200 m/s toward a substrate, where the powders are plastically deformed and bonded to the substrate, forming the layer. Unlike thermal spraying techniques, cold spraying does not include melting the powders. Like thermal spraying, cold spraying is particularly suited for depositing thin films having a thickness of about 10 µm to about 10 mm. Deposition parameters, including carrier gas, gas pressure, gas temperature, particle size, feedstock material, and nozzle design may be changed to achieve predetermined layer characteristics. A person with skill in the art would know how to adjust these process parameters.

In addition to thermal spraying processes to deposit layers of the acoustic stack, thin film deposition processes may also be used to deposit such layers. Thin film deposition processes are particularly suited for deposition of seed layers and/or thin, continuous films. Thin film deposition processes include, but are not limited to, physical vapor deposition (PVD), including electron beam physical vapor deposition (EBPVD), plasma spray, including air plasma spray (APS) and vacuum plasma spray (VPS), chemical vapor deposition (CVD), atomic layer deposition (ALD), pulsed laser deposition (PLD), molecular beam epitaxy (MBE), and electroplating.

PVD processes typically use vacuum chambers and surface interactions with precursor materials in the gas phase to form thin films. PVD may be used to deposit, for example, metals, metal carbides, metal oxides, metal alloys, silicon, silicon oxide, and any combination of any two or more thereof from solid precursor materials. In particular, source material is physically transferred in a vacuum to the substrate without any chemical reaction(s) involved. Physical vapor deposition processes include electron beam physical vapor deposition (EBPVD) and plasma spray, including air plasma spray (APS) and vacuum plasma spray (VPS). PVD is particularly suited for depositing thin films having a thickness of about 0.1 µm to about 50 µm. Deposition parameters, including pressure in the vacuum chamber, temperature of the substrate, carrier gas flow, precursor materials, and predetermined layer thickness may be changed to achieve predetermined layer characteristics. A person with skill in the art would know how to adjust these process parameters.

In contrast to PVD, CVD processes involve the reaction or decomposition of precursors on the substrate surface to form a thin film. In particular, the substrate is exposed to one or more volatile precursors, which react and/or decompose on the substrate surface to produce the desired deposit. CVD may be used to deposit, for example, metals, metal carbides, metal oxides, metal alloys, silicon, silicon oxide, and any combination of any two or more thereof from precursor gases. Generally, CVD is particularly suited to depositing thin films having a thickness of about 0.1 µm to about 50 µm. Deposition parameters, including vacuum chamber, temperature of the substrate, carrier gas flow, precursor gases, and predetermined layer thickness may be changed to achieve predetermined layer characteristics. A person with skill in the art would know how to adjust these process parameters.

ALD is a form of CVD that involves controlled alternating exposures of gaseous precursors in series of pulses such that the precursors react with the surface in a self-limiting way. ALD may be used to deposit, for example, metals, metal carbides, metal oxides, metal alloys, silicon, silicon oxide, silicon nitride, and any combination of any two or more thereof from precursor gases. ALD provides more precise thickness control and conformality than other CVD processes, and is particularly suited to depositing thin films having a thickness of about 0.1 nm to about 100 nm. Deposition parameters, including vacuum chamber, temperature of the substrate, carrier gas flow, precursor gases, and predetermined layer thickness may be changed to achieve predetermined layer characteristics. A person with skill in the art would know how to adjust these process parameters.

Electroplating (also known as electrodeposition) is a process that uses an electric current to coat a substrate with a layer of metal. Generally, electroplating includes the reduction of cations from a liquid electrolyte of the metal using electric current from an external power supply, where the substrate acts as the negative electrode of the electrolytic cell. Electroplating may be used to deposit metals and metal alloys. For example, electroplating may be used to deposit the conductive metal (e.g., nickel) layer of the acoustic stack. Electroplating is particularly suited to depositing thin films having a thickness of about 10 µm to about 1000 µm. Deposition parameters, including current density, concentration of the cations in the electrolyte, temperature, agitation, and pH of the electrolyte may be changed to achieve predetermined layer characteristics. A person with skill in the art would know how to adjust these process parameters.

Another aspect of the present technology includes methods of simultaneously forming arrays of acoustic stacks on a substrate or forming discrete layers of acoustic stacks using masks. In particular, because thermal spraying, cold spraying, and PVD are line-of-sight processes, masks may be shadow masks or sacrificial layers in a lift-off process. Lift-off processing may be used to pattern layers to deposit layers using non-line-of-sight processes, including CVD, ALD, and electroplating, to form arrays of acoustic stacks or arrays of discrete layers. These processes may be used to avoid or substantially reduce the amount of dicing used to fabricate an array of acoustic stacks.

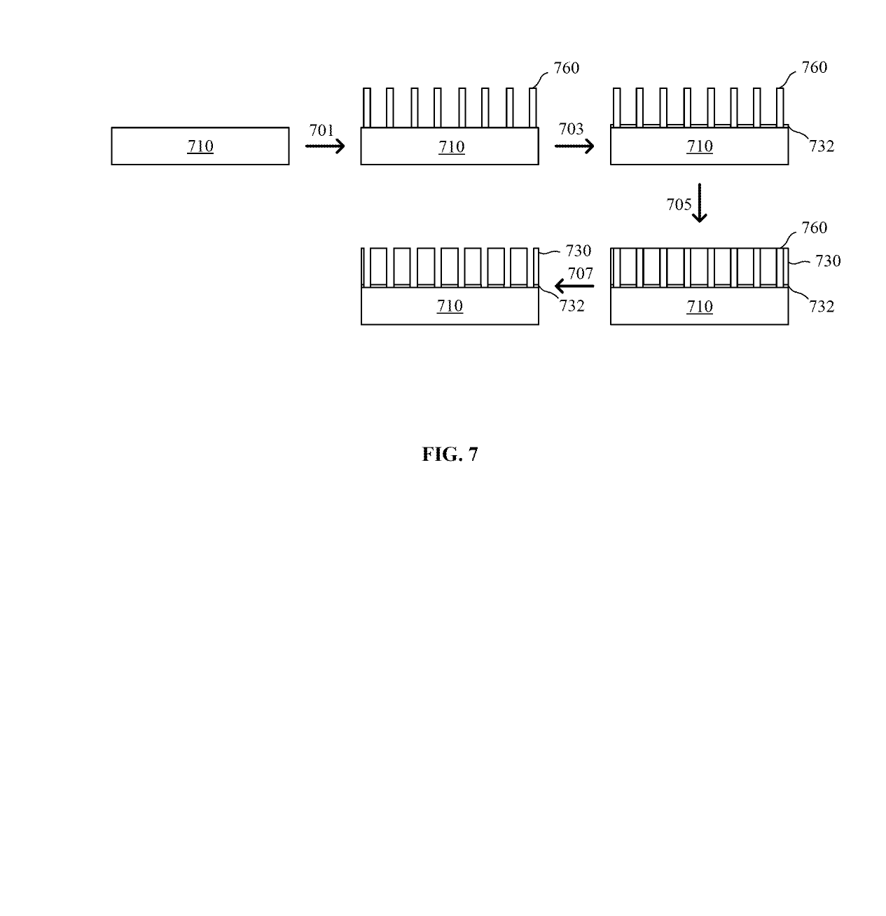

FIG. 7 is a schematic for a process for preparing a patterned dematching layer on a piezoelectric layer. In step 701, a sacrificial pattern 760 is applied to a piezoelectric layer 710. In step 703, a seed layer 732 is deposited on the piezoelectric layer 710. In step 705, the dematching layer 730 is deposited on the seed layer 732. In step 707, the sacrificial pattern 760 is lifted off (e.g., via etching or dissolution) to reveal the patterned dematching layer 730.

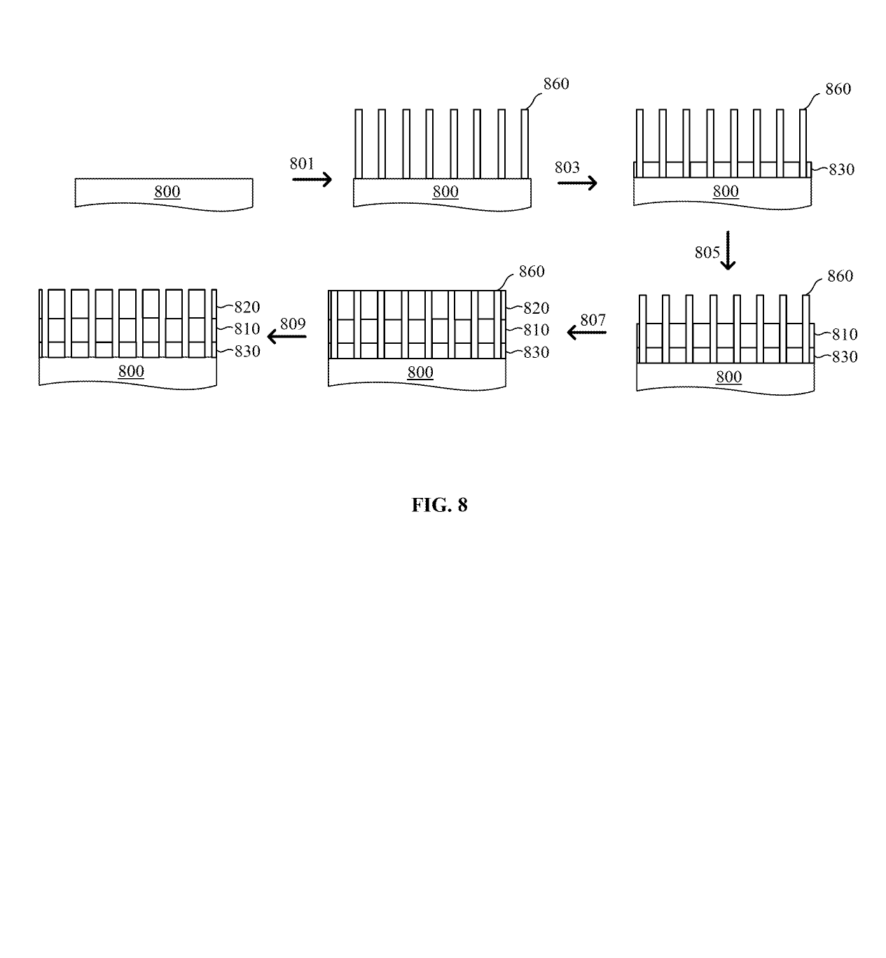

FIG. 8 is a schematic for another process for preparing a diced acoustic stack. In step 801, a sacrificial pattern 860 is applied to a substrate 800. In step 803, the dematching layer 830 is deposited on the substrate 800. In step 805, the piezoelectric layer 810 is deposited on the dematching layer 830. In step 807, the matching layer 820 is deposited on the piezoelectric layer 810. In step 809, the sacrificial pattern 860 is lifted off to reveal the array of acoustic stacks on the substrate 800.

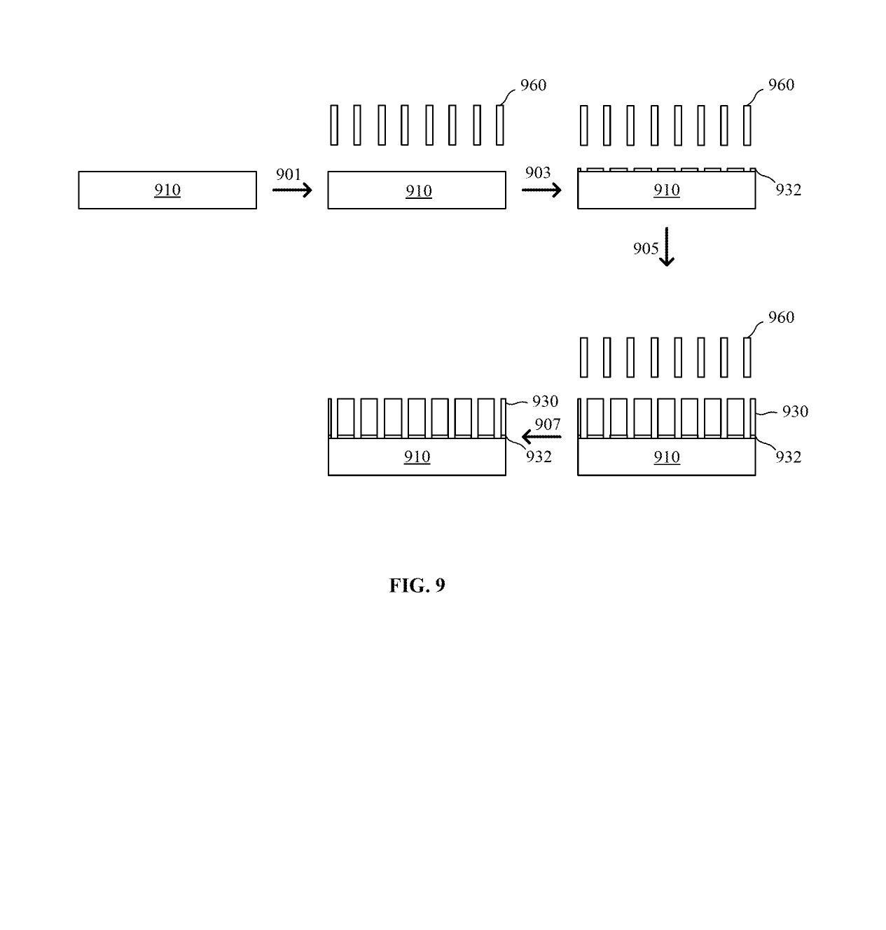

FIG. 9 is a schematic for another process for preparing a diced acoustic stack. This process uses a shadow mask for deposition of a patterned dematching layer. In step 901, a shadow mask 960 is positioned above a piezoelectric layer 910. In step 903, a seed layer 932 is deposited on the piezoelectric layer 910 using a line-of-sight technique (e.g., PVD). Because the seed layer 932 is deposited using a line-of-sight process, the shadow mask 960 imposes a pattern on the seed layer 932. In step 905, the dematching layer 930 is deposited on the seed layer 932 using a line-of-sight technique (e.g., thermal spraying). As with the seed layer 932, the dematching layer 930 is deposited using a line-of-sight process such that the dematching layer 930 has the pattern imposed by the shadow mask 960. In step 907, the shadow mask 960 is moved away from the piezoelectric layer 910, revealing the patterned dematching layer 932.

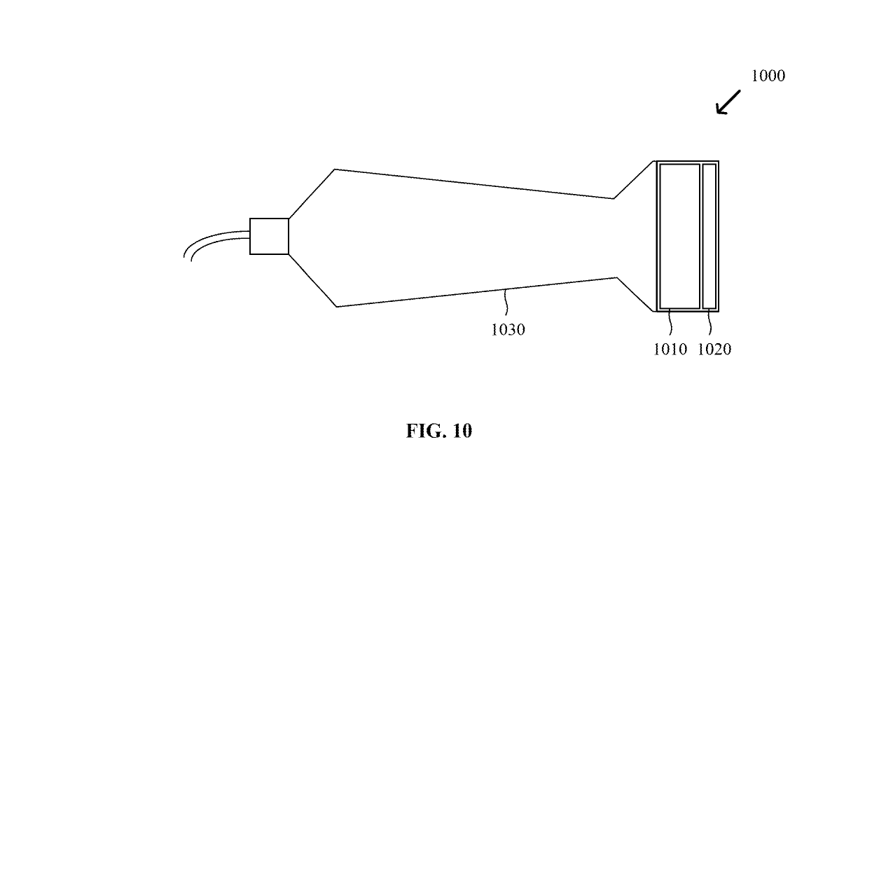

Another aspect of the present technology includes an ultrasound probe having the acoustic stack or array of acoustic stacks as disclosed herein. FIG. 10 is a cross-sectional illustration of an ultrasound probe 1000 with the acoustic stack 1010. The ultrasound probe 1000 includes a housing 1030, the acoustic stack 1010 as disclosed herein, and a lens 1020. In other embodiments, the acoustic stack 1010 is disposed within the lens 1020. The ultrasound probe 1000 can be coupled with an ultrasound medical imaging system including a processing circuit and display device. In this way, the ultrasound medical imaging system is configured to obtain ultrasound medical images of a patient using the ultrasound probe 1000 and to display the images on the display device.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes, and omissions may be made in the design, operating conditions, and arrangement of the embodiments without departing from the scope of the present disclosure as expressed in the appended claims.

What is claimed is:

1. An acoustic stack comprising:
a piezoelectric layer;
a matching layer directly disposed on a first side of the piezoelectric layer; and
a dematching layer directly disposed on a second side of the piezoelectric layer, the first side opposite the second side.

2. The acoustic stack of claim 1, wherein the acoustic stack is free of adhesive.

3. The acoustic stack of claim 1, wherein the dematching layer comprises tungsten carbide.

4. The acoustic stack of claim 3, wherein the dematching layer further comprises a seed layer disposed directly on the second side of the piezoelectric layer, the seed layer comprising gold, nickel, or aluminum.

5. The acoustic stack of claim 1, wherein the matching layer comprises:
a first matching layer comprising graphite, aluminum, silicon, polymer, or a combination of two or more thereof; and
a second matching layer comprising a polymer.

6. The acoustic stack of claim 5, wherein the matching layer further comprises a seed layer disposed directly on the first side of the piezoelectric layer, the seed layer comprising gold, nickel, or aluminum.

7. The acoustic stack of claim 5, wherein the first matching layer comprises a composite comprising graphite, aluminum, and silicon in an elemental gradation.

8. The acoustic stack of claim 1, wherein the piezoelectric layer comprises lead zirconium titanate, lead magnesium niobate, lead titanate, lead indium niobate, silicon carbide, or a combination of two or more thereof.

9. A method comprising depositing a matching layer on a first side of a piezoelectric layer via thermal spray deposition.

10. The method of claim 9, wherein depositing the matching layer comprises depositing the matching layer directly on first side of the piezoelectric layer, and thermal spray deposition comprises plasma spraying.

11. The method of claim 9, wherein depositing the matching layer comprises:
depositing a seed layer directly on the first side of the piezoelectric layer via physical vapor deposition (PVD), chemical vapor deposition (CVD), or electroplating, the seed layer comprising gold, nickel, or aluminum;
depositing a first matching layer on the seed layer via thermal spray deposition, the first matching layer comprising graphite, aluminum, silicon, polymer, or a combination of two or more thereof; and
depositing a second matching layer on the first matching layer via thermal spray deposition, the second matching layer comprising a polymer.

12. The method of claim 9, further comprising depositing a dematching layer on a second side of the piezoelectric layer via thermal spray deposition.

13. The method of claim 12, wherein depositing the dematching layer comprises depositing the dematching layer directly on the second side of the piezoelectric layer; and thermal spray deposition comprises high velocity oxygen fuel spraying (HVOF).

14. The method of claim 12, wherein depositing the dematching layer comprises:
depositing a seed layer directly on the second side of the piezoelectric layer via physical vapor deposition (PVD), chemical vapor deposition (CVD), or electroplating, the seed layer comprising tungsten carbide, gold, nickel, or aluminum; and
depositing a tungsten carbide layer on the seed layer via thermal spray deposition; and thermal spray deposition comprises high velocity oxygen fuel spraying (HVOF).

15. The method of claim 9, further comprising depositing a dematching layer on a conductive layer via thermal spray deposition.

16. The method of claim 15, wherein depositing the dematching layer comprises depositing the dematching layer directly on the conductive layer, and thermal spray deposition comprises high velocity oxygen fuel spraying (HVOF).

17. The method of claim 15, wherein depositing the dematching layer comprises:
depositing a seed layer directly on the conductive layer via physical vapor deposition (PVD), chemical vapor deposition (CVD), or electroplating, the seed layer comprising tungsten carbide, gold, nickel, or aluminum; and
depositing a tungsten carbide layer on the seed layer via thermal spray deposition; and thermal spray deposition comprises HVOF.

18. The method of claim 15, further comprising depositing the piezoelectric layer on the dematching layer via thermal spray deposition, the piezoelectric layer comprising lead zirconium titanate, lead magnesium niobate, lead titanate, lead indium niobate, silicon carbide, or a combination of two or more thereof.

19. The method of claim 9, further comprising, prior to depositing the matching layer, disposing a patterned mask on the first side of the piezoelectric layer to direct deposition of the matching layer.

20. A method of preparing an acoustic stack comprising:
plasma spray depositing a first layer on a substrate, the first layer comprising a composite comprising aluminum, silicon, and graphite;
disposing a flex circuit on the first layer;
sputtering a second layer on the flex circuit, the second layer comprising gold;
electroplating a third layer on the second layer, the third layer comprising nickel;
thermal spray depositing a fourth layer on the third layer, the fourth layer comprising tungsten carbide;
electroplating a fifth layer on the fourth layer, the fifth layer comprising nickel;
thermal spray depositing a sixth layer on the fifth layer, the sixth layer comprising a piezoelectric material;
plasma spray depositing a seventh layer on the sixth layer, the seventh layer comprising a composite comprising aluminum, silicon, and graphite; and
plasma spray depositing an eighth layer on the seventh layer, the eighth layer comprising polymethyl methacrylate.
